(19) United States
(12) Patent Application Publication
(10) Pub. No.: US 2019/0321173 A1
(43) Pub. Date: Oct. 24, 2019

(54) FLEXIBLE HEART VALVE PROSTHESIS

(71) Applicant: THE REGENTS OF THE UNIVERSITY OF CALIFORNIA, Oakland, CA (US)

(72) Inventor: Bashir Akhavan Tafti, Los Angeles, CA (US)

(21) Appl. No.: 16/469,663

(22) PCT Filed: Dec. 14, 2017
(86) PCT No.: PCT/US17/66539

§ 371 Date: Jun. 14, 2019

Related U.S. Application Data
(60) Provisional application No. 62/433,869, filed on Dec. 14, 2016.

(51) Int. Cl. A61F 2/24; A61F 2/95

(52) U.S. Cl. CPC A61F 2/2418; A61F 2/2445; A61F 2/95; A61F 2230/0067; A61F 2250/0037; A61F 2250/0039; A61F 2220/0016; A61F 2002/9528

(57) ABSTRACT

A flexible heart valve prosthetic device includes a flexible frame having multiple struts disposed radially around a central axis, each strut joined at a proximal end and a distal end, a mesh band attached to each strut between their proximal ends and their distal ends, a deployable anchoring assembly having a deployable hook positioned on each strut within the mesh band, a retrieval hook positioned at the distal and/or proximal ends of the struts, and leaflets radially disposed within the mesh band. A method of releasably anchoring a flexible heart valve prosthetic within the annulus of the heart of a subject is also disclosed.

### CROSS-REFERENCE TO RELATED APPLICATIONS

This application claims priority to U.S. provisional application No. 62/433,869 filed on Dec. 14, 2016 incorporated herein by reference in its entirety.

### BACKGROUND OF THE INVENTION

The present invention relates to medical devices and methods, particularly those that relate to the treatment of valve insufficiency, also referred to as valve regurgitation. The use of prosthetic valves delivered by traditional surgical implantation methods, or by less invasive percutaneous catheter or minimally invasive transapical methods are one possible treatment for valvar insufficiency.

The heart of vertebrate animals is divided into four chambers, and is equipped with four valves (the mitral, aortic, pulmonary and tricuspid valves) that ensure that blood pumped by the heart flows in a forward direction through the cardiovascular system. The mitral valve of a healthy heart prevents the backflow of blood from the left ventricle into the left atrium of the heart, and has two flexible leaflets (anterior and posterior cusps) that close when the left ventricle contracts. The aortic valve prevents the backflow of blood from the aorta into the left ventricle of the heart, and comprises three flexible leaflets (left, right and posterior cusps) that close after the left ventricle contracts. The pulmonary valve prevents the backflow of blood from the pulmonary artery into the right ventricle of the heart, and comprises three flexible leaflets (right, left and posterior cusps) that close after the right ventricle contracts, or after ventricular systole. The tricuspid valve prevents the backflow of blood from the right ventricle into the right atrium of the heart, and comprises two flexible leaflets (anterior and posterior cusps) that close when the right ventricle contracts. The Mitrial Valve leaflets are attached to a fibrous annulus, and their free edges are tethered by subvalvular chordae tendineae to papillary muscles in the left ventricle to prevent them from prolapsing into the left atrium during the contraction of the left ventricle.

Various cardiac diseases or degenerative changes may cause dysfunction in any of these portions of the mitral valve apparatus, causing the mitral valve to become abnormally narrowed or dilated, or to allow blood to leak (i.e. regurgitate) from the left ventricle back into the left atrium. Any such impairments compromise cardiac sufficiency, and can be debilitating or life threatening.

Numerous surgical methods and devices have accordingly been developed to treat heart valve dysfunction, including open-heart surgical techniques for replacing, repairing or reshaping the native heart valve apparatus, and the surgical implantation of various prosthetic devices such as annuloplasty rings to modify the anatomy of the native heart valve. Additionally, less invasive transcatheter techniques for the delivery of replacement heart valve assemblies have been developed. In such techniques, a prosthetic valve is generally mounted in a crimped state on the end of a flexible catheter and advanced through a blood vessel or the body of the patient until the valve reaches the implantation site. The prosthetic valve is then expanded to its functional size at the site of the defective native valve.

While these devices and methods are promising treatments for valvar insufficiency, they can be difficult to deliver, expensive to manufacture, unable to be noninvasively repositioned or removed in the event they need to be.

There have been important challenges in the development of this technology, including the complexity of the mitral valve anatomy involving a saddle oval shape, the subvalvular apparatus, the interaction with the left ventricular outflow tract (LVOT) and the aortic valve, as well as the large size of transcatheter MVI (devices and large catheters for implantation. At this stage of development, all of these limit the delivery approach to transapical in most cases.

There is a need in the art for an improved prosthetic heart valve device. The present invention meets this need.

### SUMMARY OF THE INVENTION

The present invention relates to a flexible heart valve. The valve is made up of several parts including: a deployable frame; a mesh band that forms the interface between the device and the valvular apparatus; a deployable hooking mechanisms allowing the prosthetic to securely attach to the valvular and subvalvular apparatus; and a retrieval hook allowing for the repositioning and removal of the prosthetic valve once it has been initially positioned.

In one embodiment, the deployable frame comprises a plurality of struts that attach proximally to a proximal collar and distally to a distal collar that are aligned along the central axis of the frame. In one embodiment, the frame comprises up to and including five struts; in one embodiment, the frame comprises more than five struts. In one embodiment, the plurality of struts comprises a non-ferromagnetic, flexible material. In one embodiment, the non-ferromagnetic flexible material is a shape-memory material.

In one aspect, the present invention comprises a mesh band that attaches to the deployable frame at a seam along each strut of the frame. In one embodiment, the mesh band comprises a plurality of fibers. In one embodiment the plurality of fibers comprises a non-ferromagnetic, flexible material. In one embodiment, the non-ferromagnetic, flexible material is a shape-memory material. In one embodiment, the mesh band comprises a plurality of leaflet struts designed to support the leaflet formation and coaptation and flexible tethers extending from the leaflet struts which are attached to the frame struts

In one aspect, the present invention comprises a plurality of anchoring hooks that secure the prosthetic valve to the valvular apparatus. In one embodiment, the anchoring hooks are embedded in frames that are positioned within the mesh band along struts of the deployable frame. In one embodiment, anchoring hooks are embedded in frames outside of the mesh band along the struts of the deployable frame. In one embodiment, the anchoring hooks are embeded within the mesh band in between the struts.

In one aspect, the present invention comprises a retrieval hook that attaches to the proximal collar of the deployable frame. In one embodiment, the retrieval hook comprises a rigid, non-ferromagnetic material.

In one aspect, the present invention relates to a method of removing or repositioning the flexible heart valve prosthesis by inserting a valve prosthetic removal device through the vasculature into the heart of the subject, the device comprising a fixed elongated member with a loop or hook and a reducing member. In one embodiment, the method comprises threading the loop or hook of the elongated member through the retrieval hook of the prosthetic. In one embodiment, the method comprises withdrawing the prosthetic into the reducing member by way of applying tension to the retrieval hook. In one embodiment, the method comprises removing the prosthetic device from the heart through the vasculature of the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a top perspective view of a flexible heart valve prosthesis according to one embodiment. FIG. 1B is a side view and FIG. 1C is an bottom perspective view of the flexible heart valve prosthesis shown in FIG. 1A.

FIG. 2A is a top perspective view of a flexible heart valve prosthesis having tethers according to one embodiment. FIG. 2B is a side view and FIG. 2C is an bottom perspective view of the flexible heart valve prosthesis shown in FIG. 2A. FIG. 2D is a top perspective view of the flexible heart valve prosthesis shown in FIG. 2A with leaflets of the valve in an open state.

FIG. 3A is a top perspective view of a prosthetic valve leaflet layer in a closed configuration according to one embodiment. FIG. 3B is a top perspective view of the valve of 3A in an open configuration, and FIG. 3C is a bottom perspective view of the valve of 3A in a partially open configuration.

FIG. 4A is a magnified view of a barb according to one embodiment. FIG. 4B is an isolated view of a deployable frame attached that attaches to a mesh band according to one embodiment. FIG. 4C is an isolated view of a deployable frame that attaches to a mesh band, the deployable frame having multiple leaflet struts according to one embodiment. FIG. 4D is a partial view of a deployable barb assembly in an expanded and deployed state, and FIG. 4E is a partial view of a deployable barb assembly in an un-expanded and un-deployed state. FIGS. 4F-4H show alternate views of a barb assembly according to one embodiment.

FIGS. 5A-5C are alternate magnified perspective views of a hook according to one embodiment.

FIG. 6A is a partial cutaway view of a flexible heart valve prosthesis connected to a placement device within a placement catheter according to one embodiment. FIG. 6B is a partial cutaway view of a flexible heart valve prosthesis in a partially deployed state according to one embodiment. It should be appreciated that elements not shown for illustrative purposes in this or other embodiments (such as for example mesh band, deployable barb assembly, leaflets, tethers, etc.) can be part of the flexible heart valve prosthesis as described throughout the embodiments and as would be understood by those having ordinary skill in the art.

FIG. 7A is a flow chart of a method for removing a prosthetic valve device from the heart according to one embodiment. FIG. 7B is a flow chart of a method for placing a prosthetic valve device according to one embodiment. FIG. 7C is a flow chart of a method for removing a prosthetic valve device according to one embodiment.

### DETAILED DESCRIPTION

The present invention generally relates to medical devices and implants, in particular prosthetic heart valves use to treat valve regurgitation. The prosthetic heart valve offers a design with minimal components to improve ease of placement over existing designs.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Flexible Heart Valve Prosthesis

Embodiments of the present invention provide a flexible heart valve prosthetic device that is non-invasively placed through use of a transcatheter, anchors to the annulus of the heart valve through use of a deployable barb mechanism, and has features enabling its removal in the event a repositioning or a new valve replacement is required.

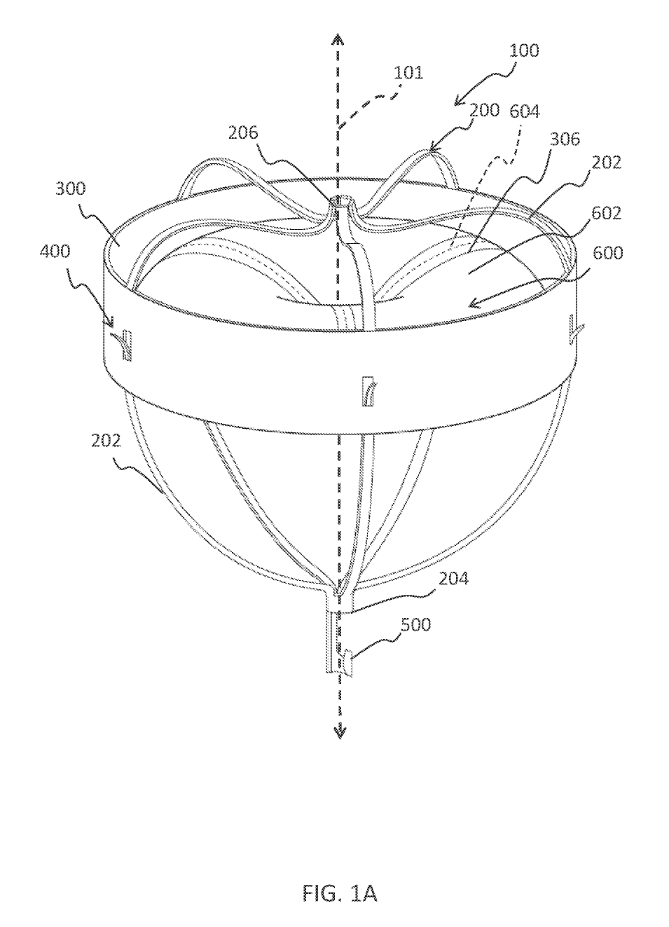

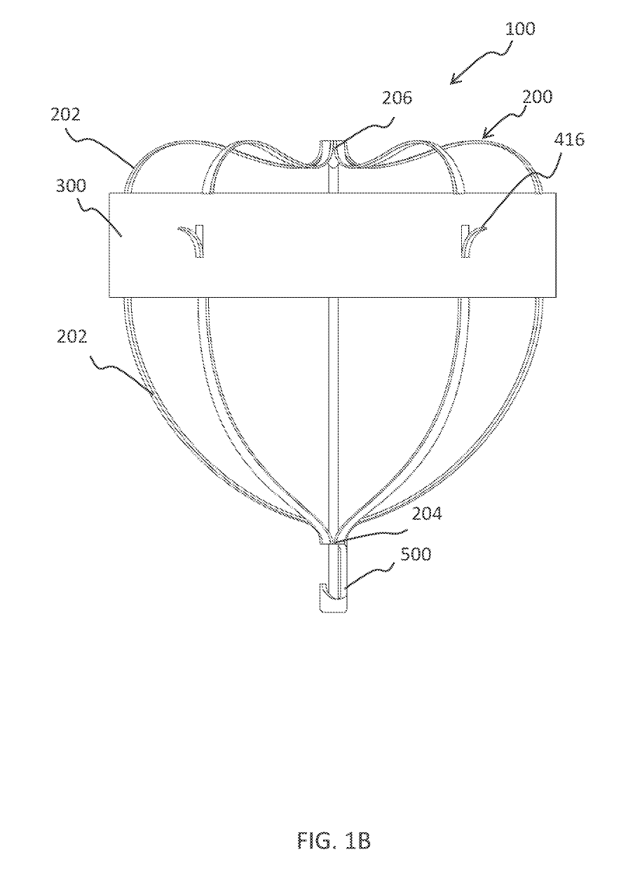

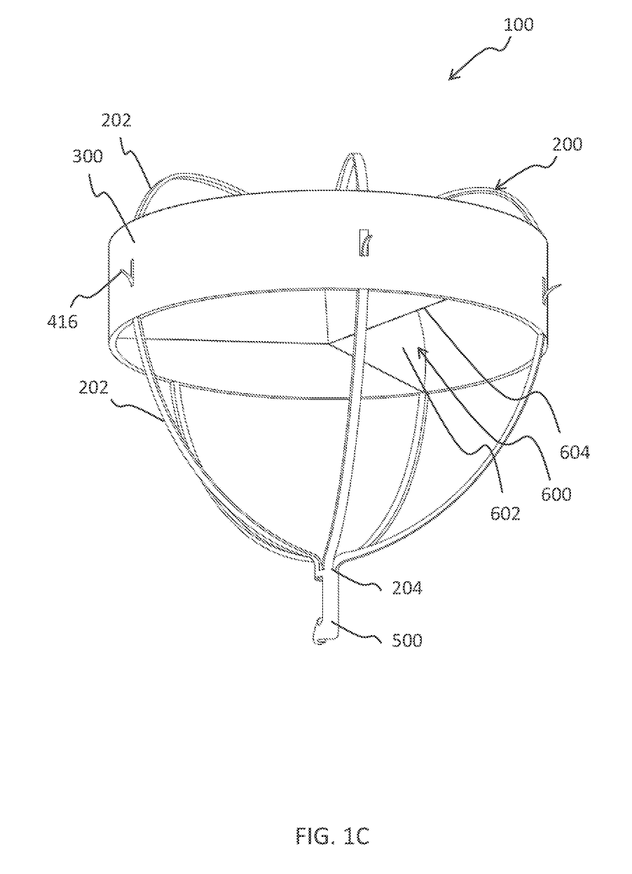

Referring now to FIGS. 1A-1C, an exemplary flexible heart valve prosthesis 100 is depicted comprising five primary structural elements: (1) a deployable frame 200; (2) a mesh band 300; (3) a deployable barb assembly 400; (4) a retrieval hook 500; and (5) leaflets 600 that attach to the frame 200.

### Deployable Frame

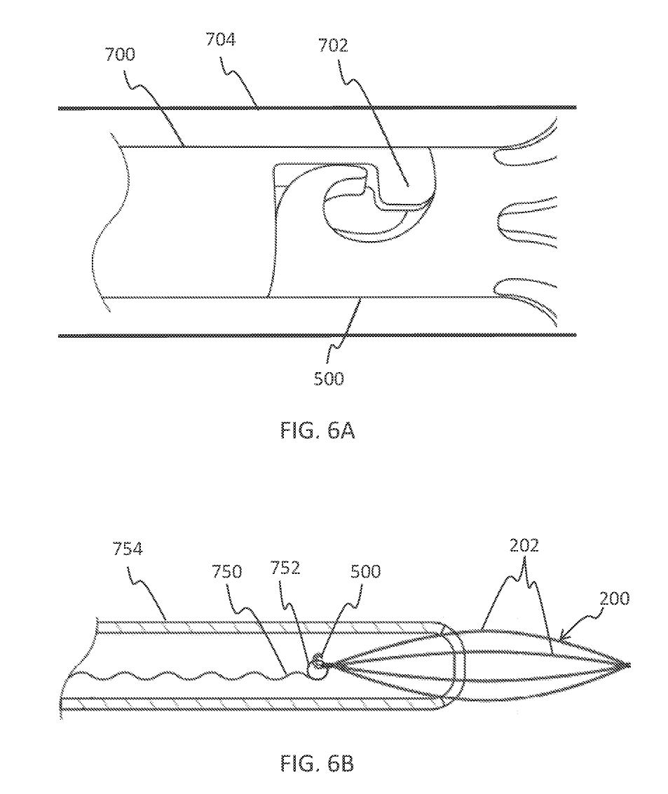

One advantageous aspect of the deployable frame 200 of the present invention is that it is less bulky and comprises minimal structural material compared to other prosthetic heart valves that are placed transcatheter. The strut design facilitates ease of collapsing the frame (see for example FIG. 6B showing the external struts in a partially collapsed state) so that the frame can fit into a delivery or retrieval catheter for placement and removal within the body. In one embodiment, the radial compression is accomplished by applying longitudinal tension to the frame drawing frame 200 into the reducing member.

The primary structural element of the flexible heart valve prosthetic 100 is the deployable frame 200. The deployable frame 200 comprises a strut structure with a plurality of struts 202 that meet at the proximal end of prosthetic 100 and are attached to a proximal collar 204. At the distal end, struts 202 meet and attach at a distal collar 206. The proximal collar 204 and distal collar 206 are aligned along a central axis 101. Also attached at the proximal end of the deployable frame 200 is a retrieval hook 500 forming the most proximal structure of the prosthetic 100.

In one embodiment, deployable frame 200 is constructed from a non-ferromagnetic, flexible, shape memory material, such as Nitinol. In one embodiment, any biocompatible, rigid, yet flexible material may be used, such as a medical grade alloy or polymer. In one embodiment, deployable frame 200 is constructed from materials such as chromium alloy, stainless steel, and titanium alloy. In one embodiment the biocompatible materials described herein may also include an anti-thrombogenic coating to prevent the incidence of embolism. The material may also include a coating comprising an immunosuppressant, e.g., rapamycin (sirolimus).

In one embodiment, a connection between one or more struts can be a rigid, a semi-rigid, or a flexible connection made by welding, sintering, or any other suitable method known in the art. In one embodiment, the connection is a movable hinge. Alternatively, two or more struts can be cut, machined, or cast from the same block of material.

In one embodiment, struts 202 are assembled to convey an expanding bias. As such, when struts 202 are compressed inwardly toward the central axis 101, an expanding bias is created, forcing the frame to return to its relaxed, expanded state when the compressive force is removed. In one embodiment, the thermally-set shape of each of struts 202 is formed to a curvature having a circumferential region of maximum diameter forming a medial band of the flexible frame. In one embodiment, the circumferential region of maximum diameter has a bias towards the distal end of prosthetic 100, the point farthest from the point of ejection from the placement catheter. In one embodiment, the circumferential region of maximum diameter has a bias towards the proximal end of prosthetic 100. In one embodiment, the circumferential region of maximum diameter has a bias towards the medial region of prosthetic 100. The struts of the frame may comprise variable stiffness to accommodate the dynamics within the beating heart. Additionally, the frame profile may be substantially formed to fit the profile of the native valve annulus (i.e. the frame designed for deployment into the mitral valve apparatus may have a substantially irregular continuous shape, or substantially D-shaped profile.

### Mesh Band

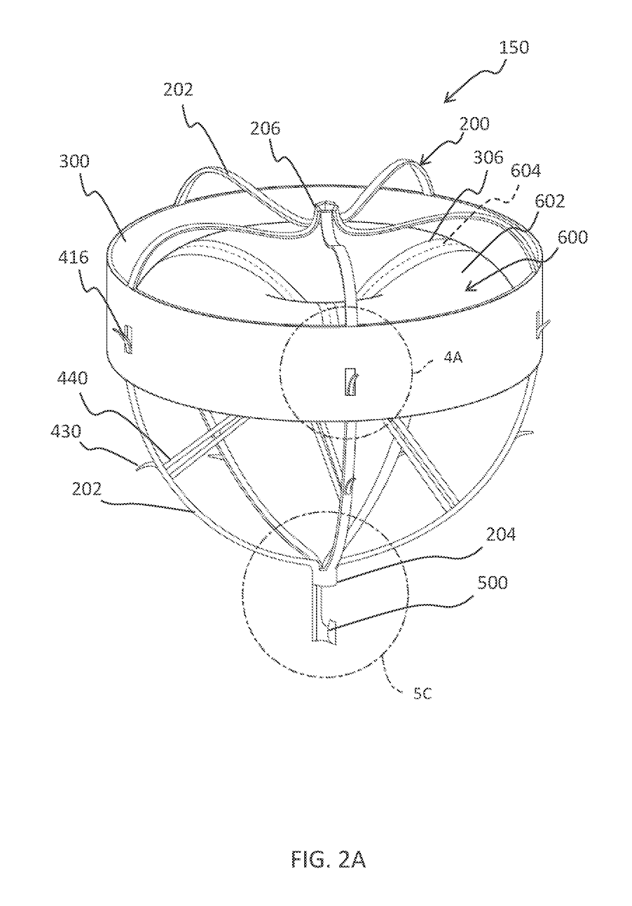

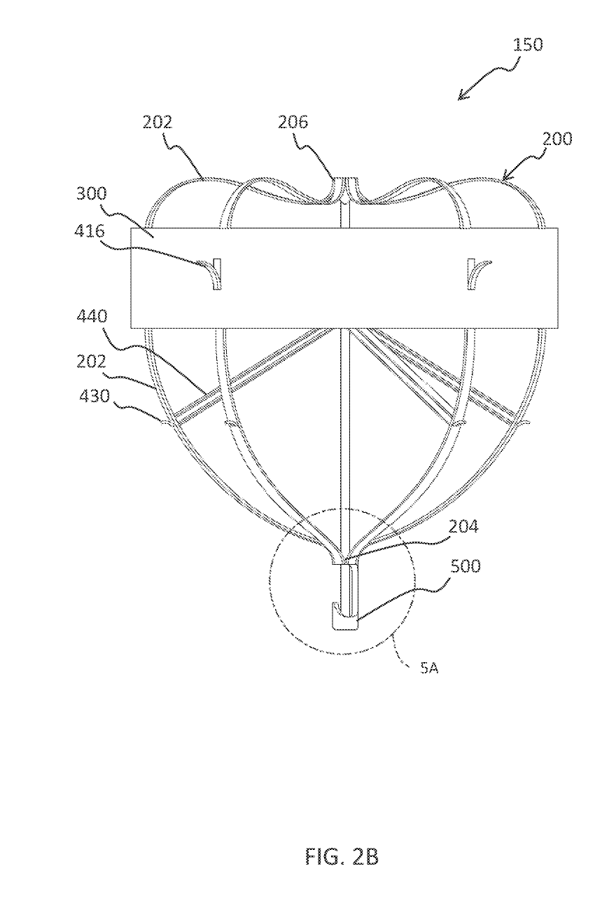

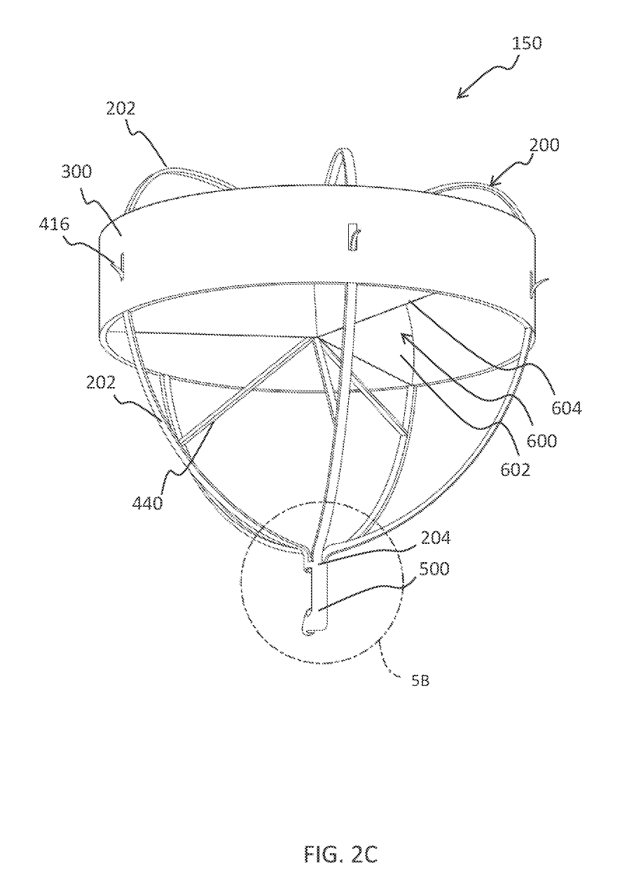

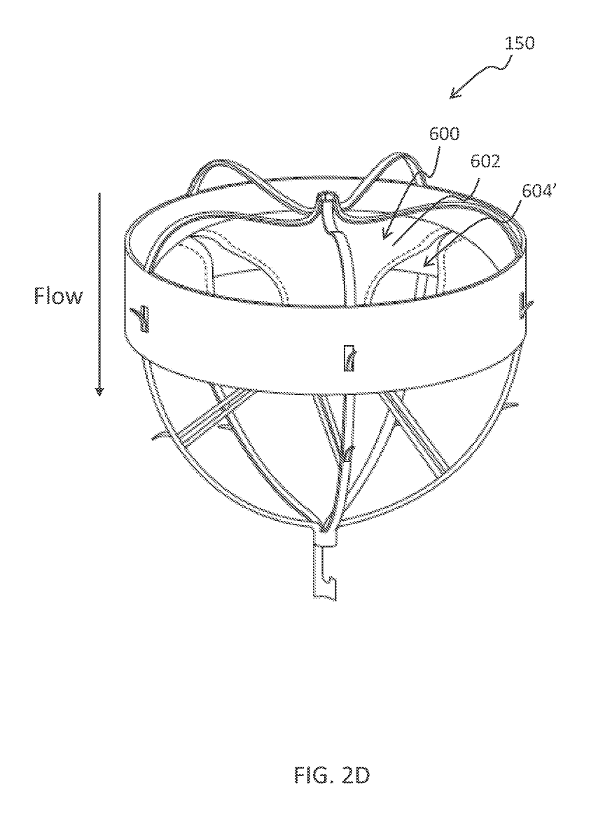

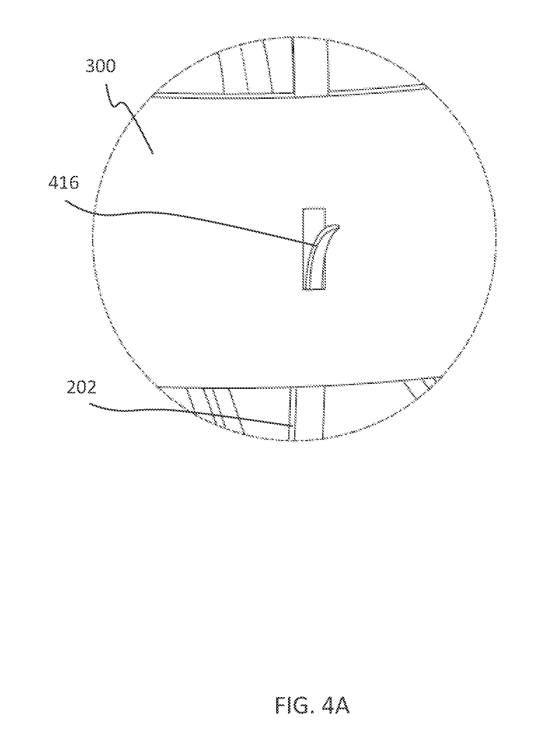

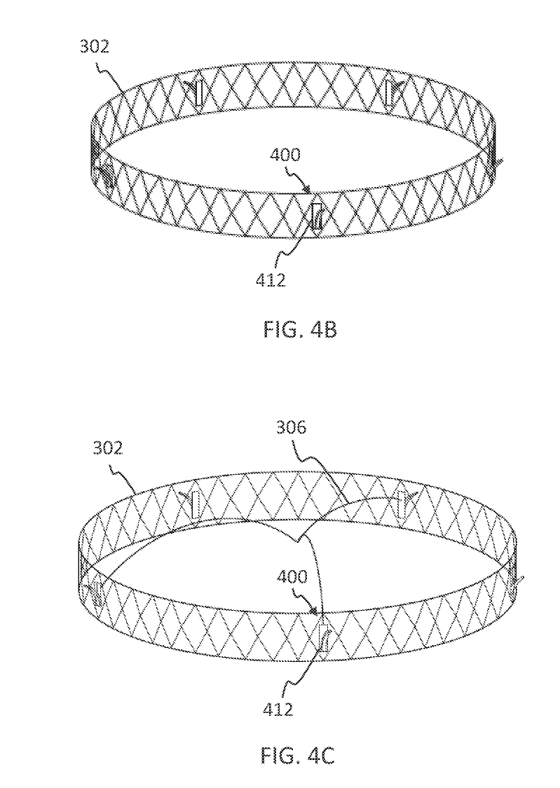

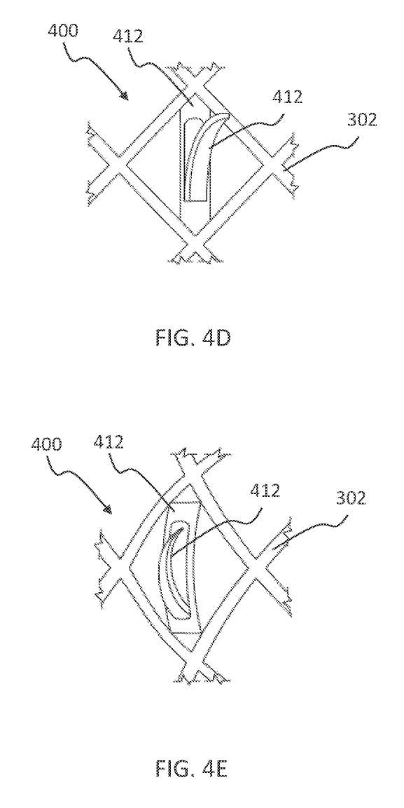

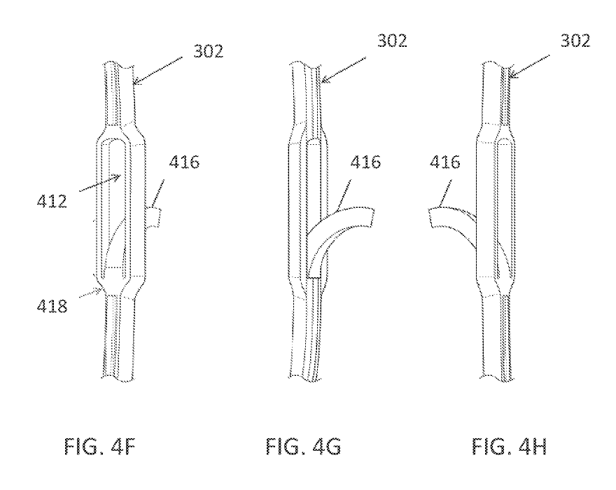

The mesh band 300 is a continuous region that forms the contact surface between the valve annulus and the prosthetic 100. As depicted specifically in FIGS. 4B and 4C, in one embodiment, the mesh band 300 comprises a plurality of crossing fibers 302. In one embodiment, the plurality of crossing fibers is composed of non-ferromagnetic biocompatible material selected from a group comprising chromium alloy, stainless steel, and titanium alloy including Nitinol. The biocompatible material is assembled into a deployable mesh according to a grid-like pattern. Mesh band 300 attaches to deployable frame 200 along the circumferential region of maximum curvature of each strut. Mesh band 300 attaches at contact seams 304 on the inner surface of mesh band 300 as depicted in FIG. 2. Mesh band 300 is compressible about axis 101 commensurate with deployable frame 200, and likewise expands in concert with the deployable frame 200 upon deployment, reinforcing the shape of mesh band 300. In one embodiment, a connection between one or more struts 202 and the mesh band 300 can be a rigid, a semi-rigid, or a flexible connection made by welding, sintering, or any other suitable method known in the art. In one embodiment, the connection is a movable hinge. In one embodiment, the connection is a rigid seam.

Mesh band 300 comprises mesh fibers 302 and anchoring barb assembly 400 securing prosthetic 100 to a valve annulus. Embedded in mesh fibers 302 are barb frames 412 for barbs 416. In one embodiment, barb frames 412 are aligned with frame strut 202 attachment points/contact seams 304, depicted in FIG. 2. Mesh band 300 comprises one or more materials with anti-thrombogenic and/or immunosuppressant properties as similar to that of deployable frame 200. In one embodiment, mesh band 300 is composed of an alloy, polymer, or any other biocompatible material that is rigid, and/or flexible, and/or elastic similar to struts 202. In one embodiment, mesh band 300 is composed of polyethylene, polyester, nylon, PTFE, ePTFE alone or in combination. In one embodiment, mesh band 300 comprises bioresorbable materials including but not limited to PLA and PGLA.

In one embodiment, mesh band 300 comprises a plurality of leaflet struts 306. Leaflet struts 306 serve as frames for leaflet attachment prior to valve placement. The leaflet struts 306 and mesh band 300 is preferably fabricated from a single piece of metallic material that has been cut so as to permit the heart valve prosthesis 100 to be compressed into a compact, generally tubular delivery configuration, and expanded into the deployment configuration further described herein. In self-expanding embodiments, the leaflet struts 306 of the prosthetic valve 100 may be fabricated from a shape-memory alloy such as a nickel-titanium alloy like nitinol, and in expandable embodiments, the leaflet struts 306 may be fabricated from any metallic material, such as chromium alloy or stainless steel, that is suitable for implantation into the body. In some embodiment, the leaflet struts may be made of polyethylene, polyester, nylon, PTFE or ePTFE.

With reference now to FIGS. 2A-2D, in one embodiment, a leaflet tether 440 can be made of the same material as leaflet struts 306 and attached to leaflet struts 306, connected at the a lower portion of struts 202 to act as a chord to provide tension support while limiting the leaflet from prolapse much like chordae tendineae. One or more leaflet tethers 440 can connect to a portion of each leaflet 602, such as the bottom tip of each leaflet 602 to bias it properly for resisting prolapse. Functionally, as illustrated for example with reference to FIGS. 2A-2D, as fluid pressure builds on the leaflets 600, the seams 604 of the leaflets 600 push open 604′ (FIG. 2D) allowing fluid to flow therethrough, and will close again (FIG. 2A) to a relaxed position when fluid flow stops. While the leaflets 600 are pushed open (FIG. 2D), leaflet tethers 440 are slacked. While the leaflets 600 are closed (FIG. 2A), the leaflet tethers 440 are taught to prevent prolapse. Since for example blood pressure in atria can periodically be much lower than that in the ventricles, the leaflets may attempt to evert to the low pressure region. The leaflet tethers 440 prevent prolapse by remaining taut and pulling the flaps, holding them in closed position. A lower barb 430 can also be introduced to a lower portion of the struts to anchor to native leaflets in the body. In some embodiments, the metallic material may be of a single thickness throughout the entirety of the strut portion, and in others may vary in thickness so as to facilitate variations in the radial force that is exerted by the anchor portion in specific regions thereof, to increase or decrease the flexibility of the anchor portion in certain regions, and/or to control the process of compression in preparation for deployment and the process of expansion during deployment.

In one embodiment, mesh band 300 may include extensions towards one end of the valve frame that extend beyond the native valve orifice. In one embodiment, the extensions protrude from a region of the prosthesis that may facilitate interaction with the native valve. In one embodiment, the extensions form a securing surface stabilizing native valve leaflets during the filling stage of the heart chamber.

### Deployable Anchoring Barbs

As depicted in FIGS. 1A-2D and 4A-4E, the prosthetic comprises a deployable anchoring barb assembly 400 that securely embeds in the heart wall. Anchoring the device into the heart wall advantageously allows the prosthetic valve leaflets 602 to open and close with fluid dynamics and heart muscle movement associated with the cardiac cycle so that the device can replicate normal valve function. The deployable anchoring barb assembly 400 comprises barbs 416 shown in FIGS. 4B and 4C incorporated into mesh band 300. The barb assembly 400 is secured to barb frame 412 which is embedded in mesh fibers 302 of mesh band 300.

Shown specifically in FIGS. 4A-4E, barb assemblies 410 or 420 can be positioned on one or more struts 202 of the deployable frame 200, such that barbs 416 point generally away from central axis 101 of prosthetic 100 and towards the annulus of the valve. Barbs 416 secure and anchor the prosthetic 100 against the valve annulus as would be understood by those skilled in the art. FIG. 4D a deployable barb assembly in an expanded and deployed state, while FIG. 4E shows a deployable barb assembly in an un-expanded and un-deployed state. Barbs 416 can be on every strut 202 or intersection/seam 302 of struts 202 and mesh band 300, or alternatively, can be present on alternate struts 202 or intersections 304 of struts 202 and mesh band 300, or any other combination of configurations. Barbs 416 may project out from the prosthetic 100 perpendicularly, or otherwise be slanted in a proximal leaning or distal leaning direction. In alternate embodiments, barbs 416 project out from the prosthetic 100 in a combination of perpendicular, proximally slanted, and distally slanted orientations. Barbs 416 in certain embodiments can protrude through a slit or slot in the mesh 300 upon expansion and deployment from the delivery catheter. In certain embodiments, while the device remains in the delivery catheter, the slit remains closed and only opens upon pressure actuation from the deploying barb and/or from expansion of the frame.

In one embodiment, barbs 416 can take a number of shapes, including curved, straight and variable thickness embodiments. Referring to the magnified views of With reference now to FIGS. 4F-4H, in one embodiment, barb assemblies 410 can be positioned also at the intersection of two or more mesh fibers 302. Barbs 416 are hinged at the bottom of barb frames 412, or at some portion further up along the openings. The hinge 418 acts as a strategic flex point so that while in a semi-compressed or compressed state, as struts 202 or mesh fibers 302 and mesh band 300 are compressed towards the central axis 101 of prosthetic 100, barbs 416 fold back about the flex point or hinge 418 and into barb frame 412, towards the central axis 101 of the prosthetic 100. In this position, barbs 416 remain tucked in below the outer surface of mesh band 300 (see also FIG. 4E). The hinge 418 can be created structurally, for instance by the removal or reduction of framing material (e.g. formation of opening of frame 412), creating a point of flexion along the frame 412. Alternatively, the hinge 418 can be created by a manufacturing step that incorporates a less rigid material at the desired flexion point, or by the introduction of additives that reduce material rigidity at the flexion point. Another method of forming the hinge 418 includes a mechanical joint connecting two or more moving parts. Alternate embodiments do not have a hinge 418, and otherwise feature a contiguous member and composition of material along the length of the struts 202 and the mesh fibers 302. Minimal exposure of barbs 416 above the surface of the prosthetic 100 while in the semi-compressed and compressed states facilitates smooth advancement and retraction of the prosthetic 100 during loading, placement and retrieval procedures. Advantageously, prosthetic valves according to embodiments of the present invention can fit into smaller delivery and retrieval catheters and devices, providing for greater ease of placement and retrieval for those skilled in the art of placement of such devices.

### Retrieval Hook

The retrieval hook feature is largely advantageous over standard heart valve prostheses that traditionally required open-heart procedures to remove and generally offer no opportunities for repositioning once initially placed by catheter or open procedure.

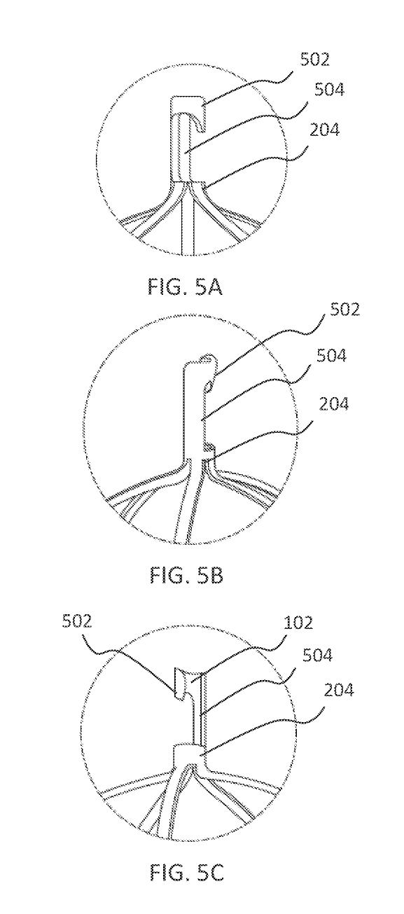

Referring for example to FIGS. 1A-2D and FIGS. 5a-5c, prosthetic heart valve retrieval hook assembly 500 is depicted. Retrieval hook 500 attaches to prosthetic 100 at proximal collar 204 and comprises a hollow channel 102 through which placement catheter guidewire (not shown) can extend through the center of prosthetic 100 in line with central axis 101. Hook body element 504 terminates in hook 502 which can be grasped and pulled in a manner that reintroduces prosthetic 100 into a placement catheter (not shown) resulting in a compressed state allowing one skilled in the art to retrieve prosthetic 100.

In one embodiment, applying tension to hook 502 withdraws it into a retrieving catheter comprising a reducing member. This tension compresses struts 202 and mesh band 300 into compact, tubular conformation allowing for retrieval, replacement or repositioning of prosthetic heart valve 100.

As illustrated in FIG. 6A, a placement device 700 including a placement hook 702 can releasably attach to the retrieval hook for placement or retrieval of the prosthetic 100. The placement device can be sized to fit within the lumen of a placement or retrieval catheter 704. With reference to FIG. 6B, a snare 750 having a loop 752 at its distal end can be used in placement or retrieval of the prosthetic 100. It should be appreciated that elements not shown in FIG. 6B for illustrative purposes (such as for example mesh band, deployable barb assembly, leaflets, tethers, etc.) are part of the prosthetic 100 and are also collapsible. Advantageously, the prosthetic 100 is shaped to easily slide and collapse within the lumen of a catheter. Further, collapsing the device by sliding pulling the prosthetic into the lumen of a catheter advantageously retracts the barbs, causing less trauma to the patient during placement and removal procedures. Deploying the device by pushing it out of the lumen benefits from these same advantages, enhancing ease of placement while minimizing trauma to the patient. The flexibility and small profile of the device during placement and removal benefits both the medical professional performing the procedure and the patient.

### Leaflets and Valve Structures

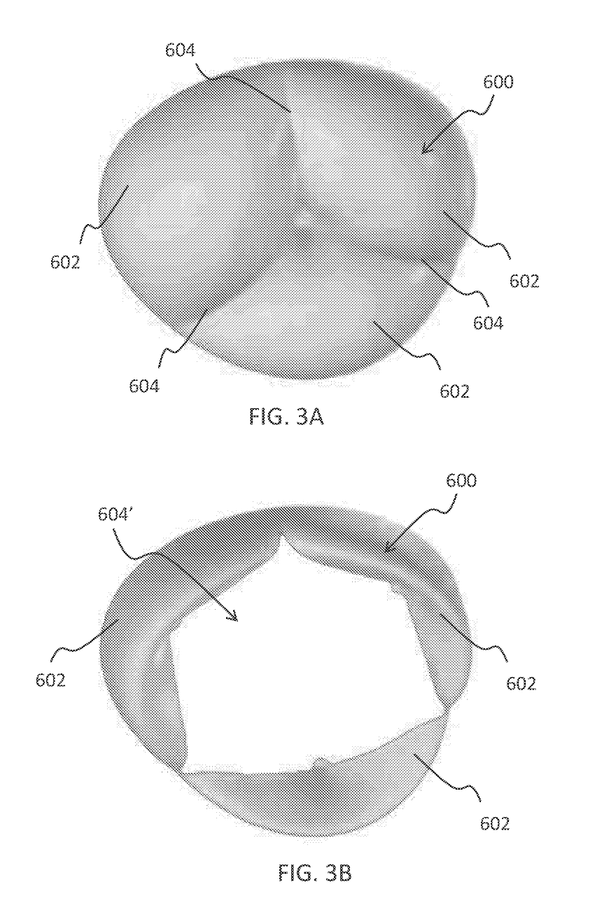

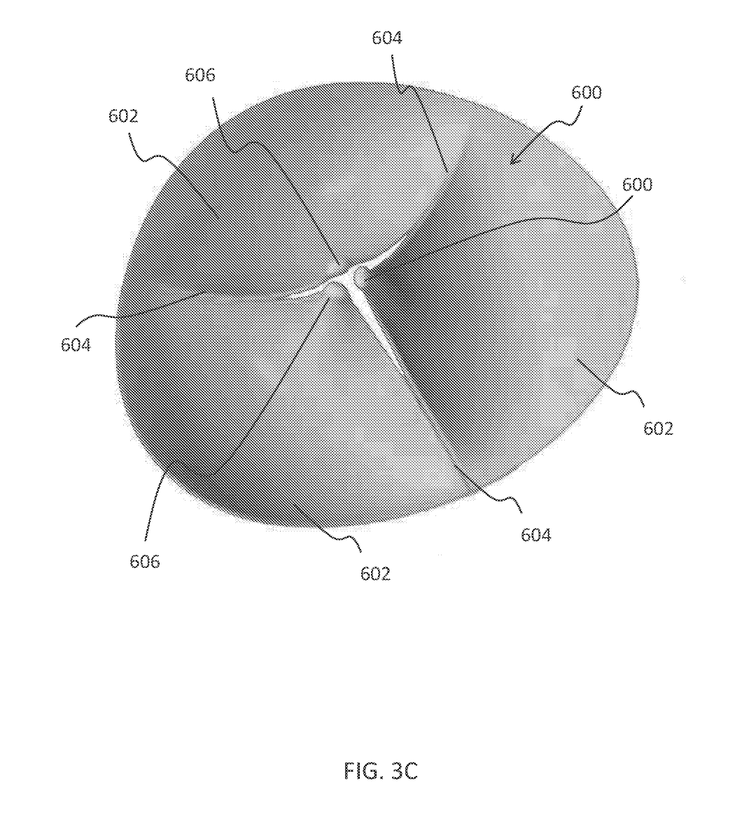

Referring now to FIGS. 3A-3C, the prosthetic valve 100 may have an open configuration (FIG. 3B) in which the prosthetic valve leaflets 602 are disposed away from one another and the seams 604′ are open, and a closed configuration (FIG. 3A) in which the prosthetic valve leaflet seams 604 engage one another. Blood flows freely through the prosthetic valve 100 and leaflets 600 in the open configuration, and retrograde blood flow across the prosthetic valve 100 is substantially prevented in the closed configuration. The prosthetic valve 100 reduces or eliminates valve regurgitation. The prosthetic valve 100 may comprise a therapeutic agent, and may elute the therapeutic agent from the prosthetic valve 100 into adjacent tissue.

The plurality of prosthetic valve leaflets 602 may comprise a tricuspid or bicuspid leaflet configuration. It should be appreciated that there is no limitation to the number of leaflets used. At least a portion of the one or more prosthetic valve leaflets 602 may comprise tissue or a synthetic material. As shown in certain embodiments, one or more of the plurality of prosthetic valve leaflets 602 may be disposed over one or more leaflet struts 306 that are radially biased inward relative to the mesh band 300. In one embodiment, the one or more leaflet struts may comprise one or more suture holes extending therethrough and that may be sized to receive a suture for attaching valve leaflets 602 to leaflet struts 306. As shown specifically in FIG. 3C, an anchoring point 606 having extra material, an opening, or some other special structure can be used to anchor tethers as described in various embodiments.

The leaflets 602 may be fabricated from a single piece or from multiple pieces of standard biologic prosthetic materials, such as cryo- or chemically-preserved pericardium (e.g. bovine, equine, porcine, human), or from standard suitable synthetic prosthetic materials (e.g. fiber-reinforced matrix materials) well known in the art, and may be sewn or otherwise adhered to the leaflet struts 306 to form the valve leaflets 602 in any standard suitable manner.

The leaflets 602 may also include chordae, wherein longitudinal fibers attach the leaflets to the frame. The chordae may be composed of any flexible biocompatible material, including metallic and polymer fibers.

### Methods of Placement

Standard practice dictates that generally once a prosthetic valve is placed, whether minimally invasively through a procedure such as TAVR/TAVI or through an open-heart procedure, in the event that a replacement is needed, an invasive open-heart procedure is required. Alternatively, a new valve is placed over top of the preexisting one. Both of these procedures present complications for the patient and complications for the surgeon. However, prosthetic 100 of the present invention offers a device and procedure for safely removing the placed valve and implanting a replacement prosthetic without requiring an open-heart procedure.

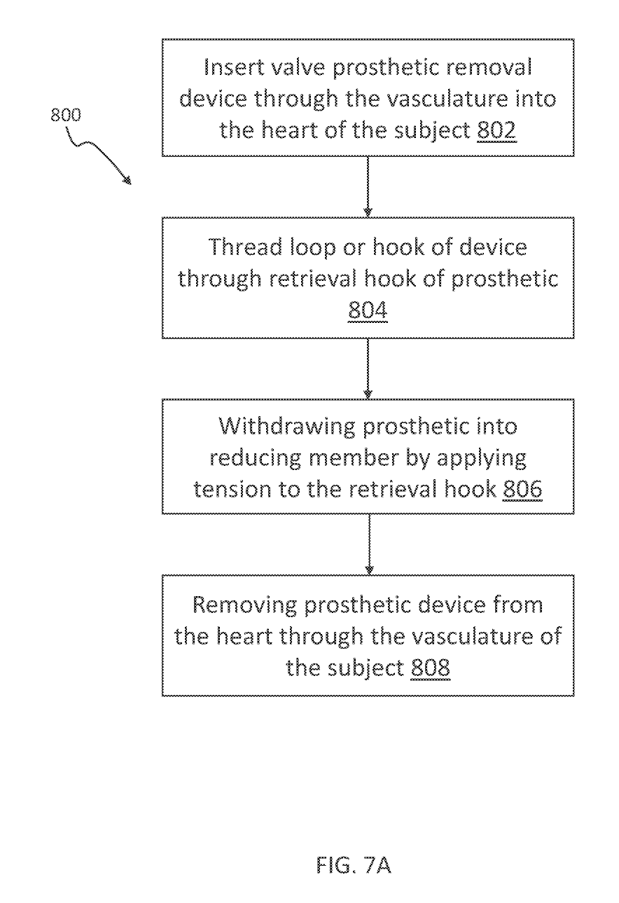

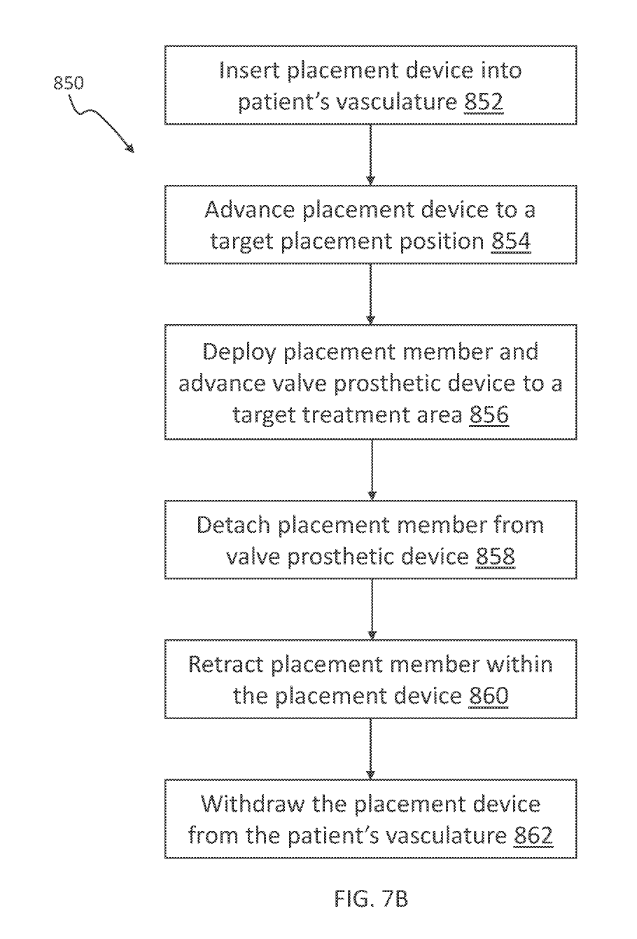

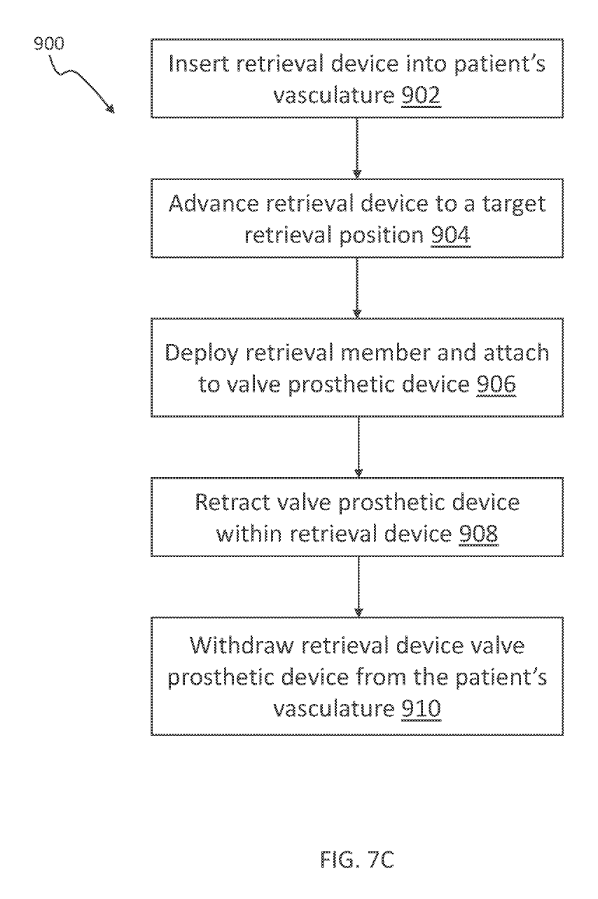

With reference now to FIG. 7A, a method of removing the flexible heart valve prosthesis 100 according to one embodiment includes the steps of inserting the valve prosthetic removal device through the vasculature into the heart of the subject 802, threading the loop or hook of the device through retrieval hook of prosthetic 804, withdrawing the prosthetic into reducing member by applying tension to the retrieval hook 806, and removing the prosthetic device from the heart through the vasculature of the subject 808. More specifically, the method can include the steps of inserting the compressed heart valve contained within the placement catheter (not shown), guiding the placement catheter into the orifice of the appropriate valve of the heart to be replaced, ejecting the valve 100 from the placement catheter by partially releasing tension on prosthetic 100 in its compressed state thereby unsheathing prosthetic from placement catheter, verifying that mesh band 300 is appropriately aligned with heart valve annulus, releasing remainder of tension or fully unsheathing prosthetic heart valve prosthetic 100 resulting in full expansion to resting state of: deployable frame 200, mesh band 300, and anchoring barbs 400. In one embodiment, the placement guidewire utilizes hook 500 to guide placement and deployment of prosthetic 100. Upon release of tension, frame 200 and mesh band 300 deploy to their resting state against a heart annulus. As frame 200 and band 300 deploy, barb assembly 400/hooks 416 actuate and embed in wall of heart once frame 200 and mesh band 300 reach their resting fully deployed state. In the event prosthetic 100 requires repositioning following initial deployment, tension can be achieved by engaging retrieval hook 500 with a placement catheter and withdrawing frame into the placement catheter in order to reduce diameter of frame 200 and mesh band 300. The device compresses to a tubular conformation. One skilled in the art can then reposition and redeploy prosthetic 100 so that frame 200 and mesh band 300 are more suitably located, and anchoring barb assembly 400 then secure prosthetic into a new position. In one embodiment, the valve prosthetic is delivered to the mitral valve by use of catheter via a trans-venous approach by crossing the septal wall from right atrium to left atrium and initially positioned near the annulus of the mitral valve. The placement catheter may include unsheathing a tubular member to expose barb assembly 400/hooks 416 and frame 200. The prosthetic 100 can be completely exposed or partially exposed outside the catheter when hooks 416 are allowed to engage the valve annulus, or the left atrial wall. Movement of the placement catheter can provide control of the prosthesis during the deployment. In one embodiment, prosthetic 100 can be delivered to the mitral valve via flexible catheter in a transfermoal approach, crossing the aortic valve into the left ventricle and the mitral valve orifice. In one embodiment, two flexible catheters can be used wherein a first catheter is placed via transvenous approach and a second catheter is placed via transfemoral approach. One flexible catheter is used to hold hook 500 and apply tension to the catheter introduced through the septal wall. The two-catheter method can provide a more stable and controlled deployment and accurate positioning of the prosthesis across the mitral annulus.

With reference now to FIG. 7B, a method of insertion according to one embodiment includes the steps of inserting a placement device into the patient's vasculature 852, advancing the placement device to a target placement position 854, deploying the placement member and advancing the valve prosthetic device to a target treatment area 856, detaching the placement member from the valve prosthetic device 858, retracting the placement member within the placement device 860, and withdrawing the placement device from the patient's vasculature 862.

With reference now to FIG. 7C, a method of removal according to one embodiment includes the steps of inserting the retrieval device into patient's vasculature 902, advancing the retrieval device to a target retrieval position 904, deploy the retrieval member and attaching to the valve prosthetic device 906, retracting the valve prosthetic device within retrieval device 908, and withdraw the retrieval device valve prosthetic device from the patient's vasculature 910.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A flexible heart valve prosthetic device, comprising:
a flexible frame comprising a plurality of struts disposed radially around a central axis, each strut joined at a proximal end and a distal end;
a mesh band attached to each strut between their proximal ends and their distal ends;
a deployable anchoring assembly comprising a deployable hook positioned on each strut within the mesh band;
a retrieval hook positioned at the proximal and/or distal ends of the plurality of struts; and
a plurality of leaflets radially disposed within the mesh band.

2. The device of claim 1, wherein the plurality of struts of the deployable frame deploys away from the central axis of the device.

3. The device of claim 1, wherein the deployable frame is constructed from a non-ferromagnetic, flexible material.

4. The device of claim 3, wherein the non-ferromagnetic, flexible material is selected from the group consisting of: a chromium alloy, stainless steel, a titanium alloy, and Nitinol.

5. The device of claim 1, wherein the deployable frame comprises a coating with an anti-thrombolytic or immunosuppressant agent.

6. The device of claim 1, wherein the mesh band comprises a plurality of fibers.

7. The device of claim 1, wherein the mesh band comprises a plurality of leaflet struts.

8. The device of claim 7, where the leaflet struts are constructed from non-ferromagnetic, flexible material.

9. The device of claim 8, wherein the non-ferromagnetic, flexible material is is selected from the group consisting of: a chromium alloy, stainless steel, a titanium alloy, and Nitinol.

10. The device of claim 7, where the leaflet struts are constructed from flexible material such as polyethylene, polyester, nylon, PTFE, and ePTFE.

11. The device of claim 6, wherein the mesh band is constructed from a non-ferromagnetic biocompatible material.

12. The device of claim 11, wherein the non-ferromagnetic biocompatible material is selected from the group consisting of: a chromium alloy, stainless steel, a titanium alloy, and Nitinol.

13. The device of claim 1, wherein the plurality of deployable hooks is constructed from a non-ferromagnetic biocompatible material.

14. The device of claim 13, wherein the non-ferromagnetic biocompatible material is selected from the group consisting of: a chromium alloy, stainless steel, a titanium alloy, and Nitinol.

15. The device of claim 1, wherein the retrieval hook is constructed from a non-ferromagnetic biocompatible material.

16. The device of claim 15, wherein the non-ferromagnetic biocompatible material is selected from the group consisting of: a chromium alloy, stainless steel, a titanium alloy, and Nitinol.

17. The device of claim 1, wherein the plurality of leaflets comprises two or three leaflets.

18. The device of claim 17, where the plurality of leaflets is constructed from a biological material or a synthetic material.

19. The device of claim 18, wherein the biological material is preserved biological tissue collected from a donor wherein the donor species is selected from the group consisting of human, bovine, equine, and porcine.

20. The device of claim 18, wherein the synthetic material is a fiber-reinforced matrix material.

21. A method of releasably anchoring a flexible heart valve prosthetic within the annulus of the heart of a subject, the method comprising:
inserting a valve prosthetic removal device through the vasculature into the heart of the subject, the device comprising a fixed elongated member with a loop or hook and a reducing member;
threading the loop or hook of the elongated member through the retrieval hook of the prosthetic of claim 1;
withdrawing the prosthetic into the reducing member by way of applying tension to the retrieval hook; and
removing the prosthetic device from the heart through the vasculature of the subject.

22. The method of claim 21, wherein tension upon the retrieval hook reversibly retracts the plurality of deployable hooks whereby releasing the device from its anchored position.

23. The method of claim 21, wherein tension upon the retrieval hook reversibly compresses the device into a tubular conformation.
